(19) United States
(12) Patent Application Publication
(10) Pub. No.: US 2003/0004143 A1
(43) Pub. Date: Jan. 2, 2003

(54) Use of NSAIDs for prevention and treatment of cellular abnormalities of the female reproductive tract

(76) Inventors: Christopher P. Prior, Rosemont, PA (US); Drore Eisen, Cincinnati, OH (US); Louis Herlands, Cambridge, MA (US)

Correspondence Address: CHERYL H AGRIS PHD, PO BOX 806, PELHAM, NY, 10803, US

(21) Appl. No.: 10/125,218

(22) Filed: Apr. 18, 2002

Related U.S. Application Data
(60) Provisional application No. 60/284,756, filed on Apr. 18, 2001.

(51) Int. Cl. A61K 31/60; A61K 31/415; A61K 31/405; A61K 31/192

(52) U.S. Cl. 514/165; 514/406; 514/420; 514/570

(57) ABSTRACT

The invention is directed to uses of non-steroidal anti-inflammatory drugs (NSAIDs) for the treatment and prevention of cellular abnormalities of the female reproductive tract.

This application claims priority from U.S. provisional application serial No. 60/284,756, filed Apr. 18, 2001, the contents of which are incorporated herein by reference.

1. FIELD OF THE INVENTION

The invention is directed to topical uses of non-steroidal anti-inflammatory drugs (NSAIDs) for the treatment and prevention of cellular abnormalities of the female reproductive tract, wherein said NSAIDs are selected from the group consisting of COX-1 selective cyclooxygenase inhibitors, nonselective cyclooxygenase inhibitors, and partially selective cyclooxygenase-2 (COX-2) inhibitors.

2. BACKGROUND OF THE INVENTION

2.1 Cervical cancer

Cervical cancer is the third most common type of cancer in women with an average age of 45 years at time of diagnosis. Predisposing factors include multiple sexual partners, early onset of sexual activity and early childbearing, sexually transmitted diseases, specifically, human papillomavirus (HPV or genital warts), HIV infection, and genital herpes. HPV infects the vulva, vagina & cervix (reviewed in O'Shaughnessy et al., 2002, Clin. Cancer Res. 8:314-346). It is generally accepted that HPV is involved in the pathogenesis of cervical intraepithelial neoplasia (CIN) and cervical cancers. Women who were exposed to the drug Diethylstilbestrol in utero are also at risk for developing rare vaginal and cervical cancers and many other abnormalities of the uterine, cervical and vaginal tissues. Cervical cancer typically develops in a well-defined and gradual fashion over many years. The precursor lesion to invasive cervical cancer, CIN (cervical intraepithelial neoplasia) is also known as SIL (squamous intraepithelial lesion) of the cervix. SILS are classified as low-grade (LGSIL) and high-grade (HGSIL).

Initial subtle changes occur in the superficial cells of the cervix and these abnormalities may result in dysplasia, a premalignant condition of the cervix. The dysplasia may progress to preinvasive cancer (carcinoma in situ) which invades only the outer layer of the cervix. If left untreated, the cancerous cells may spread to the deeper layers, and ultimately, to the other pelvic organs and other distant sites. The Pap smear, which shows abnormal cells, dysplasia, or cervical cancer, is a very effective method of detecting abnormal cells. Therefore, a routine pelvic examination, including a Pap smear is recommended yearly beginning at the onset of sexual activity, or by the age of 20 in non-sexually active women. This screening process is critical since the early stages of cervical cancer produce no symptoms. The most common abnormal Pap result is atypical squamous cells of undetermined significance (ASCUS) and each year in the US over two million pap tests are reported as ASCUS. ASCUS may progress to dysplasia and no drugs have been approved to treat ASCUS and low grade cervical dysplasia in order to prevent progression to later stage disease. By contrast, invasive cancer frequently results in symptoms as the tumor invades the underlying tissue causing bleeding and pain. Studies have demonstrated that early detection and treatment of precancerous and early stage cervical cancer has a dramatic impact on survival. For example, the 5 year survival rate approximates 85% for Stage I cervical cancer and only 12% for Stage 4.

Treatment options depend on the stage of the disease and consist of surgery, (localized excision of precancer or superficial cancers vs. radical hysterectomy for invasive cancer). Some experts consider it a reasonable standard of care to follow LGSILS and HPV infection, because only 12% progress to cervical carcinoma in situ. A cone biopsy, which consists of removing a wedge of the cervix for biopsy, may be all that is required to remove all of the malignant cells of a surface cancer. Cryosurgery (destruction of tissue by freezing), laser therapy, and surgical excision via loop diathermy are also used frequently to treat preinvasive cancer. Nearly 40% of patients with CIN/SILs have multifocal lesions involving two or three sites on the female genital tract. Treating the vagina and vulva with surgical ablation often results in disfiguration and sexual dysfunction and is characterized by a relatively high rate of reoccurrence. Radiation therapy, chemotherapy, radical surgery or a combination of the three may be employed to treat cervical cancer. However, there are of course morbidity and side effects associated with each of these therapies. Therefore, there is a need for other therapies that result in fewer and less radical side effects or can be used in conjunction with surgery, radiation or chemotherapy.

2.2. NSAIDs

Though NSAIDs have been classically categorized according to their chemical structure, since the discovery of COX-2, they are increasing being categorized according to their COX-2 selectivity. Categories of NSAIDs are usually referred to: COX-1 selective, nonselective, COX-2 preferential and COX-2 selective. COX-1 selective NSAIDs include but are not limited to flurbiprofen, ketoprofen, fenoprofen, piroxicam and sulindac. Nonselective inhibitors include but are not limited to aspirin, ibuprofen, indomethacin, ketorolac, naprosen, oxaprosin, tenoxicam and tolmetin. Relatively COX-2 selective inhibitors include but are not limited to diclofenac, etodolac, meloxicam, nabumetone, nimesulide and 6-MNA. Highly selective COX-2 inhibitors include: celecoxib, rolfecoxib and other drugs like L-743337, NS-398 and SC 58125.

Experimental work has shown that increased amounts of prostaglandins and COX-2, a key enzyme involved in the prostaglandin synthetic pathway, are commonly found in a wide range of premalignant tissues and malignant tumors including cervical dysplasia and cancer.

Extensive evidence from genetic and pharmacological studies indicate that COX-2 is mechanistically linked to the development of cancer (see, for example, Dannenberg et al., The LANCET Oncology 2:544-551 (2002).

Elevated prostaglandin and COX-2 levels substantially contribute to carcinogenesis by inhibiting apoptosis (see Tsujii and DuBois Cell 83, 493-501 (1995)) and stimulating angiogenesis (see Tsujii et al., Cell 83, 493-501 (1995); Williams et al., J. Clin Invest 105, 1589-94 (2000) and Masferrer et al., Cancer Research 60, 1306-11 (2000).

Although there is substantial evidence that overexpression of COX-2 is linked to tumorigenesis, it is not clear whether the antitumor effects of NSAIDs entirely result from the inhibition of COX-2 activity. COX-independent mechanisms as ell as the inhibition of COX-1 may contribute to the antitumor effect of NSAIDs. COX-independent mechanisms of NSAIDS have been the subject of a recent workshop (see Hwang et al. Neoplasia 2, 91-97 (2002).

Highly selective cyclooygenase-2 inhibitors have been developed to minimize gastric side effects that can occur when non-selective COX inhibitors are administered peroral. However, there is no data that demonstrates their superiority over selective cyclooxygenase-1 inhibitors, nonselective cyclooxygenase inhibitors, and partially selective cyclooxygenase-2 (COX-2) inhibitors for the treatment and prevention of cancer. To the contrary, emerging evidence indicates that COX-1 activity may contribute to carcinogenesis, so that maintaining pan-COX inhibition is likely to be more beneficial than selectively inhibiting COX-2. For example, the knocking out of the COX 1 gene also protects against the formation of intestinal and skin tumors in the Min mouse model. See Chulada et al. (Cancer Research; 60: 4705-08 2000) Thus, inhibiting both COX-1 and COX-2 is likely to be more beneficial than inhibiting COX-1 alone. However, in spite of this COX-1 knockout study, few if any researchers in the field have emphasized the potential role of COX-1 in carcinogenesis.

The growth of tumors typically is also associated with immune suppression. During the development of tumors, host monocytes and macrophages are triggered to produce high levels of prostaglandin E₂ (PGE₂) which has immunosuppressive effects. The immunosuppressive effects of PGE₂ include the inhibition of: T and B lymphocyte proliferation, lymphokine production, cytotoxicity of natural killer (NK) cells, effector functions of T-cells, B-cells, and macrophages, and generation of cytotoxic T lymphocytes and lymphokine-activated killer (LAK) cells.

Studies using prostaglandin synthesis inhibitors, like non-steroidal anti-inflammatory drugs (NSAID), have provided further evidence for the role of prostaglandins in mediating immuno suppression. Considerable evidence further suggests that NSAIDs may have an important role in chemoprevention. The use of NSAIDs has been shown effective in reducing or inhibiting tumor growth and bone metastasis.

For example, Hong et al., 2000, “Cyclooxygenase regulates human oropharyngeal carcinomas via the proinflammatory cytokine IL-6: a general role for inflammation?” FASEB J. 14:1499-1507 discloses that ketorolac, a pan-COX inhibitor and NSAID continuously and significantly decreased PGE₂ production and IL-6 and IL-8 levels in all OPC cell lines tested and reduced OPC growth in vivo but not in vitro. It is noted that ketorolac does play a role in decreasing inflammation. It is speculated that chronic inflammation may play a role in promoting the development of OPC and that perhaps the mechanism may apply to other epithelial tumor systems modulated by COX activity. Falk et al., U.S. Pat. Nos. 6,136,793, 6,114,314, 6,103,704 and 6,218,373 disclose compositions suitable for topical application to the skin in the form of a gel or awn comprising nonsteroidal antinflammatory drugs (NSAIDs) and an effective amount of hyaluronic acid sufficient to transport the drug to a site of a disease or condition. NSAIDs disclosed included diclofenac, inomethacia, naproxen, tromethamine salt of ketorolac, ibuprofen, piroxicam propionic acid derivatives, acetylsalicylic acid and finixin. These compositions are disclosed to be used to treat basal cell carcinoma, actinic keratoses lesions, fungal lesions, “liver” spots, squamous cell tumors, metastatic cancer of the breast to the skin, primary and metastatic melanoma in the skin, genital warts, cervical cancer, human papilloma virus of the cervix, psoriasis, corns of the feet and hair loss in pregnant women. It is thought that NSAID prevents the enzymatic production of prostaglandins, which block macrophage and Natural Killer (NK) cell functions in the local anti-tumor immune response. The hyaluronic acid is thought to act by transporting the NSAID with it until the space between the cells to the area of trauma. As a result, it enhances the activity of prostaglandin synthesis inhibition and reduces any side effects that are associated with the use of the NSAID.

Wechter et al., U.S. Pat. No. 5,955,504 has disclosed the use of R-NSAIDs primarily for the treatment and prevention of colorectal cancers. However, large amounts of R-NSAID need to be used.

Cavanaugh, Re36,419 primarily discloses a method for using an NSAID and in particular, ketorolac for treatment of primary or recurring squamous cell carcinomas of the oral cavity or oropharynx by topical administration.

Seibert et al., U.S. patent application publication US 2001/0047024 A1 discloses a method of using cyclooxygenase-2 inhibitors in the treatment and prevention of neoplasia. In this publication, the term “cyclogenase-2 inhibitor” denotes a compound able to inhibit cyclogenoxygenase-2 without significant inhibition of cycloxygenase-1.

Fey et al., U.S. Patent application publication US 2001/0011097 discloses a method of reducing or inhibiting mucositis by administering, for example, an NSAID in combination with an inflammatory cytokine inhibitor, mast cell inhibitor, an MMP inhibitor or an NO inhibitor.

### 3. OBJECTS OF THE INVENTION

It is an object of the invention to provide an effective means to prevent and treat cellular abnormalities of the female reproductive tract that can be used alone or in conjunction with existing treatments.

It is a further object of the invention to provide a means for administering the NSAID to a mammal which results in high local concentration of the drug in the target tissue with minimal systemic (blood) concentration of said NSAID.

It is a further object of the invention to provide a means for administering the NSAID to a mammal that results in minimal exposure to the gastric epithelium so as to provide for reduced incidence of gastrointestinal toxicity including gastrointestinal hemorrhage.

### 4. SUMMARY OF THE INVENTION

The invention is directed to methods of using a anti-inflammatory drug (NSAID), essentially free of hyaluronic acid to topically treat or prevent a cellular abnormality of one or more organs of the reproductive tract of a female mammal comprising administe ring to said reproductive tract in need thereof an amount of a non-steroidal anti-inflammatory drug (NSAID), essentially free of hyaluronic acid alone or as an adjunct to chemotherapy, surgery and/or radiation therapy effective to treat or prevent said cellular abnormality. The invention is also directed to compositions comprising said NSAID wherein said composition is essentially free of hyaluronic acid and the use of said compositions for treating or preventing said cellular abnormality, a cellular abnormality of one or more organs of the reproductive tract of a female mammal. The invention is also directed to preventing metastases and secondary cancers from developing after removal of the first tumor with said NSAIDs and/or compositions. As will be described in further detail below, the NSAID is preferably a COX-1 selective cyclooxygenase inhibitor, a non-selective cyclooxygenase inhibitor, or a partially selective cyclooxygenase-2 (COX-2) inhibitor. The invention is further directed to the use of said NSAIDs for use in the manufacture of a medicament for prevention or treatment of said cellular abnormality or for preventing said metastases or secondary cancers.

The systemic administration of high doses of NSAIDs, including the new generation of highly selective COX-2 inhibitors (like celecoxib), may cause adverse events such as ulcer complications, atria; fibrillation, and cardiac arrhythmia (Scrip #2610 p12 (Feb. 21, 2001). Therefore, in order to minimize gastric as well as systemic exposure and maximize delivery to the site of action, the concept of using an NSAID-containing topical application was developed.

Local or topical delivery of NSAIDs is likely to achieve high local concentrations of NSAIDS in the target tissue. In a specific embodiment, the amount of NSAID administered topically is sufficiently high to access COX-independent pathways of antitumor effects. Other advantages of topical delivery of COX-1 selective cyclooxygenase inhibitors, nonselective cyclooxygenase inhibitors, and partially selective cyclooxygenase-2 (COX-2) inhibitors include reduced GI and systemic toxicity and improved therapeutic index. The concentration of the drug is high in the target tissue and the concentration in the bloodstream and the GI tract is minimized. Sufficient uptake to the mucosal epithelium is achieved without the use of hyaluronic acid or other carrier.

In addition, with topical administration, a much higher concentration of NSAID in the diseased tissue is obtained than when the NSAID is systemically administered. The levels of NSAID in the diseased tissue obtained through topical or local administration of NSAID enables accessing both COX-dependent and COX-independent antitumor effects. Furthermore, levels of NSAID sufficient to access certain COX-independent pathways would be impossible to achieve through systemic administration of the NSAID.

As defined herein, “essentially free of hyaluronic acid” means that the NSAID or composition comprising said NSAID does not contain any amounts of hyaluronic acid significant to affect the transport of the NSAID. The composition comprises preferably preferably less than about 0.1% hyaluronic acid, and most preferably, those comprising less than about 0.01% hyaluronic acid.

“Treat” and “treatment”, as used herein, mean to attempt to slow the progress of or to reverse the symptoms of the condition being addressed.

### 5. DETAILED DESCRIPTION OF THE INVENTION

The method of the present invention is directed to using NSAIDs to treat and prevent cellular abnormalities of the reproductive tract of a female mammal. In a specific embodiment, the female mammal is a human patient.

5.1. NSAIDs

The NSAIDs used in the method of the present invention may be a COX-1 selective cyclooxygenase inhibitor, a nonselective cyclooxygenase inhibitor, or a partially selective cyclooxygenase-2 inhibitor. As defmed herein, a “nonselective cyclooxygenase inhibitor” has a selectivity ratio of cyclooxygenase-2 inhibition over cyclooxygenase-1 inhibition of between about 0.1-15. Alternatively, the NSAIDs may be partially selective cyclooxygenase-2 (COX-2) inhibitors. As defined herein, a “partially selective cyclooxygenase-2 inhibitor” has a selectivity ratio of cyclooxygenase-2 inhibition over cyclooxygenase-1 inhibition of between 15-50. As defined herein, a “COX-1 selective cycloxygenase inhibitor” has a selectivity ratio of cyclooxygenase-2 inhibition over cyclooxygenase-1 inhibition of less than 0.1. The NSAIDs may include but are not limited to flurbiprofen, ketoprofen, fenoprofen, carprofen, diflunisal, piroxicam and sulindac, aspirin, ampyrone, ibuprofen, indomethacin, ketorolac, naprosen, niflumic acid, oxaprosin, suprofen, tenoxicam, tamoxifen, ticlopidine, tenidap, tolmetin, diclofenac, etodolac, flufenamate, meclofenamate, mefenamic acid, meloxicam, nabumetone, nimesulide, resveratrol,, 6-MNA, zomepirac, and tomoxiprol, and mixtures thereof.

In a preferred embodiment, the NSAID is ketorolac. “Ketorolac”, as used herein, is (.+−.)5(benzoyl)-2,3-dihydro-1H-pyrrolizine-1carboxylic acid, and the pharmaceutically acceptable non-toxic esters and salts thereof, as disclosed in U.S. Pat. No. 4,089,969 issued to Muchowski & Kluge on May 16, 1978. The (−)-S enantiomer of ketorolac is preferred.

Pharmaceutically acceptable esters of ketorolac include but are not limited to, alkyl esters derived from hydrocarbons of branched or straight chain having one to about 12 carbon atoms. Examples of such esters are methyl, ethyl, propyl, isopropyl, butyl, t-butyl, isoamyl, pentyl, isopentyl, hexyl, octyl, nonyl, isodecyl, 6-methyldecyl and dodecyl esters.

Pharmaceutically acceptable salts of ketorolac include salts derived from either inorganic or organic bases. Salts derived from inorganic bases include sodium potassium, lithium ammonium, calcium, magnesium, ferrous, zinc, copper, manganese, aluminum, ferric, manganic salts and the like. Particularly preferred are the ammonium, potassium, sodium, and lithium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, piperidine, tromethamine, dicyclohexylamine, choline and caffeine.

The preferred ketorolac salt, which is soluble in the composition of the subject invention in which it is incorporated, for use in the compositions and methods of the present invention is the racemic mixture of (+)R and (−)-S enantiomer of ketorolac tromethamine, and most preferred is its (−)-S enantiomer, (−)-5(benzoyl)-2,3-dihydro-1H-pyrrolizine-1-carboxylic acid, 2-amino-2-(hydroxymethyl)-1,3-propanediol.

5.2 Compositions

One aspect of t he present invention is compositions comprising a safe and effective amount, preferably from about 0.001% to about 15%, 0.003% to about 10%, more preferably from about 0.005% to about 1%, more preferably still from about 0.01% to about 0.5%, even more preferably from about 0.1% to about 0.5%, still more preferably from about 0.05% to about 0.2% ketorolac, and a pharmaceutically acceptable topical carrier. The compositions can be in the form of creams, foams, lotions, ointments, douches (solutions or dissolvable powders), suppository, gels (time release) or sprays. The carrier or vehicle diluent can be aqueous or non-aqueous, for example alcoholic or oleaginous, or a mixture thereof, and may additionally contain surfactants, emollients, lubricants, stabilizers, dyes, perfumes, antimicrobial agents either as active ingredients or as preservatives, and acids or bases for adjustment of pH. The preferred pH is about 4 to 5. Conventional methods are used in preparing the compositions.

In a preferred embodiment, the compositions and NSAIDs are administered topically. Specifically, the NSAIDs may be administered via an applicator syringe, sponge, suppository, or foam dispensing unit. For topical applications, the pharmaceutically acceptable carrier may additionally comprise organic solvents, emulsifiers, gelling agents, moisturizers, stabilizers, other surfactants, wetting agents, preservatives, time release agents, and minor amounts of humectants, sequestering agents, dyes, perfumes, and other components commonly employed in pharmaceutical compositions for topical administration. In particular, the NSAIDs may be administered to the mucosal lining of the reproductive tract.

Alternatively, NSAIDS may be delivered via an elastomer or polymer ring composed of silicone or other material that is inserted and stays in place in the upper portion of the vagina, where it releases NSAID at an effective concentration providing a consistent low-dose of NSAID for an extended period of time. This period of time can range from days to as much as three months.

The NSAIDs of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

NSAIDs of the present invention may additionally be combined with chemotherapeutic agents or further antimicrobial (antiviral, antibacterial or antifungal) compounds to provide a combination therapy. Combination therapy is intended to include any chemically compatible combination of an NSAID of the present invention with other compounds of the present invention or other compounds outside of the present invention, as long as the combination does not eliminate the activity of the NSAID of the present invention. For example, one or more NSAIDs of the present invention may be combined with other antiviral agents, antimicrobial agents, or chemotherapeutic agents. In certain embodiments of the invention, a compound of the present invention is used in combination with one or more other therapeutic agents, such as anti-inflammatory, anti-viral, anti-fungal, amoebicidal, trichomonocidal, analgesic, anti-neoplastic, anti-hypertensives, anti-microbial and/or steroid drugs, to treat antiviral infections. In certain preferred embodiments, viral infections are treated with a compound of the present invention in combination with one or more of beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, , acyclovir, amantadine, rimantadine, recombinant soluble CD4 (rsCD4), nevirapine, cidofovir (Vistide™), trisodium phosphonoformate (Foscarnet™), cyclovir, famcyclovir, pencyclovir, valacyclovir, nucleic acid/replication inhibitors, interferons, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof.

Combination therapy can be sequential, that is the treatment with one agent first and then the second agent, or it can be treatment with both agents at the same time. The NSAIDs and the second agent may be combined into one composition. The sequential therapy can be within a reasonable time after the completion of the first therapy before beginning the second therapy.

The compositions of the present invention should be administered at least one per day but may be administered up to three times daily. The specific treatment regimen will be dependent on the nature of the condition being treated. The treatment may be for as short as three months and could continue for up to five years.

The compositions of the present invention may be held in the female reproductive tract for a period of from 15 seconds to about 24 hours. The compositions of the present invention should have favorable tissue residence times. The time the drug persists in the tissue is the residence time. A favorable residence time is a time that allows for convenient dosing and maintains sufficient tissue drug concentration to inhibit the COX enzymes.

5.3 Uses

The NSAIDs and compositions of the present invention may be used to treat cellular abnormalities of the organs of the reproductive tract of a female mammal, preferably a female human patient. Such organs include but are not limited to the vagina, vulva and cervix. In a particular embodiment, the NSAIDs and compositions of the present invention are administered in a therapeutically effective dose to prevent or treat neoplasia, or to treat or prevent cervical intraepithelial neoplasia. In a specific embodiment, the NSAIDs and compositions of the present invention may be administered in an amount effective to induce apoptosis, In yet another specific embodiment, the NSAIDs and compositions of the present invention are administered in a dose sufficient to access COX-independent pathways of antitumor effects

In a specific embodiment, the NSAIDs and compositions of the present invention may be used to treat cellular abnormalities present in the cervix. These include but are not limited to HPV positive infected cells, atypical squamous cells of undetermined significance, also called “ASCUS” , Cervical Intraepithelial Neoplasia (CIN I), moderate dysplasia (CIN II) and severe dysplasia (CIN III) and squamous intraepithelial lesions (“SIL”), carcinoma in situ, and cervical cancer, as well as primary and recurring squamous cell carcinomas.

The NSAIDs may also be used to prevent metastases in those patients suffering from cervical cancer. Furthermore, the NSAIDs may be used to prevent unrelated secondary tumors from developing in patients where primary tumors have been removed from the reproductive tract.

The specific embodiments herein disclosed are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A method for prevention or treatment of a cellular abnormality of one or more organs of the reproductive tract of a female mammal comprising topically administering to said reproductive tract in need thereof an amount of a non-steroidal anti-inflammatory drug (NSAID), alone or as an adjunct to chemotherapy, surgery and/or radiation therapy effective to treat said cellular abnormality, wherein said NSAID is selected from the group consisting of COX-1 selective inhibitors, non-selective cyclooxygenase inhibitors and partially selective cyclooxygenase-2 inhibitors.

2. The method according to claim 1, wherein said cellular abnormality is selected from the group consisting of HPV infected cells, atypical squamous cells of undetermined significance (ASCUS), intraepithelial neoplasia, hyperplasia, dysplasia, carcinoma in situ, and carcinoma.

3. The method according to claim 1, wherein said organ is selected from the group consisting of the cervix, vagina and vulva.

4. The method according to claim 1 wherein the NSAID is selected from the group consisting of flurbiprofen, ketoprofen, fenoprofen, carprofen, diflunisal, piroxicam and sulindac, aspirin, ampyrone, ibuprofen, indomethacin, ketorolac, naprosen, niflumic acid, oxaprosin, suprofen, tenoxicam, tamoxifen, ticlopidine, tenidap, tolmetin, diclofenac, etodolac, flufenamate, meclofenamate, mefenamic acid, meloxicam, nabumetone, nimesulide, resveratrol,, 6-MNA, zomepirac, and tomoxiprol, and mixtures thereof.

5. The method according to claim 1 in which the NSAID is administered in an amount sufficiently high to access COX-independent pathways of antitumor effects.

6. The method according to claim 1 wherein the NSAID is ketorolac.

7. The method according to claim 6 wherein the ketorolac is ketorolac tromethamine.

8. The method according to claim 6 wherein the ketorolac is an S-enantiomer of ketorolac tromethamine.

9. The method according to claim 1, wherein the mammal is a human.

10. The method according to claim 1, wherein the NSAID is administered to mucosal lining of said reproductive tract.

11. A method for treating an abnormality of one or more organs of the reproductive tract of a female mammal comprising topically administering to said reproductive tract in need thereof an amount of a composition comprising about 0.001% to about 10%, by weight of an NSAID, essentially free of hyaluronic acid alone or as an adjunct to surgery and/or radiation therapy effective to treat said cellular abnormality, wherein said NSAID is selected from the group consisting of COX-1 selective inhibitors, non-selective cyclooxygenase inhibitors and partially selective cyclooxygenase-2 inhibitors.

12. The method according to claim 11, wherein said composition is in the form of a douche, spray, foam, cream, ointment, or suppository.

13. The method according to claim 12, wherein the composition is held in the female reproductive tract for a period of from 15 seconds to about 24 hours.

14. The method according to claim 11, wherein said composition is in the form of an elastomeric ring.

15. The method according to claim 14, wherein the composition is held in the female reproductive tract for a period of at least about two days.

16. The method according to claim 14, wherein the composition is held in the female reproductive tract for a period of up to about three months.

17. A method for treatment of a cellular abnormality of the cervix of a female mammal comprising topically administering to said reproductive tract in need thereof an amount of an NSAID alone or as an adjunct to chemotherapy, surgery and/or radiation therapy effective to treat said cellular abnormality, wherein said NSAID is selected from the group consisting of COX-1 selective inhibitors, non-selective cyclooxygenase inhibitors and partially selective cyclooxygenase-2 inhibitors.

18. The method according to claim 17, wherein said cellular abnormality of the cervix is selected from the group consisting of HPV positive cells, atypical squamous cells of undetermined significance, mild cervical dysplasia, moderate cervical dysplasia, severe cervical dysplasia, squamous intraepithelial lesions and primary and recurring squamous cell carcinomas.

19. A method for the prevention of a primary or recurring squamous cell carcinoma of one or more organs of the reproductive tract of a female mammal comprising topically administering to said reproductive tract in need thereof an amount of an NSAID, alone or as an adjunct to chemotherapy, surgery and/or radiation therapy effective to treat said cellular abnormality, wherein said NSAID is selected from the group consisting of COX-1 selective inhibitors, non-selective cyclooxygenase inhibitors and partially selective cyclooxygenase-2 inhibitors.

20. The method according to claim 19, wherein said organ is the cervix.

21. A composition comprising an NSAID and a second agent which is a chemotherapeutic agent or antimicrobial agent, wherein said NSAID is selected from the group consisting of COX-1 selective inhibitors, non-selective cyclooxygenase inhibitors and partially selective cyclooxygenase-2 inhibitors and wherein said composition is essentially free of hyaluronic acid.

22. The composition according to claim 21, wherein the second agent is an antiviral agent, antibacterial or antifungal agent.

23. The composition according to claim 21, wherein said composition is in the form of a douche, spray, foam, cream, ointment or suppository or elastomeric ring.

24. A method for the prevention of an unrelated tumor developing in a patient having a primary tumor of the reproductive tract of a female mammal, wherein said tumor has been excised, comprising topically administering to said reproductive tract in need thereof an amount of an NSAID alone or as an adjunct to chemotherapy, surgery and/or radiation therapy effective to prevent said secondary tumor, wherein said NSAID is selected from the group consisting of COX-1 selective inhibitors, non-selective cyclooxygenase inhibitors and partially selective cyclooxygenase-2 inhibitors.

25. A method for preventing metastases in a female mammal having a primary tumor of the reproductive tract comprising administering to said mammal in need thereof comprising topically administering to said reproductive tract in need thereof an amount of an NSAID alone or as an adjunct to chemotherapy, surgery and/or radiation therapy effective to prevent said metastases, wherein said NSAID is selected from the group consisting of COX-1 selective inhibitors, non-selective cyclooxygenase inhibitors and partially selective cyclooxygenase-2 inhibitors.
